(19) United States
(12) Patent Application Publication
(10) Pub. No.: US 2026/0309661 A1
(43) Pub. Date: Oct. 8, 2026

(54) MICROFLUIDIC INTEGRATED CMOS BASED IMPEDANCE ARRAY BIOSENSOR

(71) Applicants: Nam-seog Kim, Cheongju-si (KR); UNIVERSITY OF UTAH RESEARCH FOUNDATION, Salt Lake City, UT (US)

(72) Inventors: Nam-seog Kim, Cheongju-si (KR); Jungkyu Kim, Salt Lake City, UT (US)

(21) Appl. No.: 19/483,030

(22) PCT Filed: Jun. 6, 2024
(86) PCT No.: PCT/US2024/032858

§ 371 Date: Nov. 10, 2025

Related U.S. Application Data
(60) Provisional application No. 63/471,375, filed on Jun. 6, 2023.

(51) Int. Cl. G01N 27/02; G01N 33/543

(52) U.S. Cl. CPC G01N 27/028; G01N 33/5438

(57) ABSTRACT

A point-of-care diagnostic device configured to measure characteristics of a biological sample. The point-of-care device includes an electronic component package and a complementary metal-oxide-semiconductor (CMOS) biosensor formed on the electronic component package. The CMOS biosensor includes a fluidic system, an electrode array in fluid communication with the fluidic system, and an impedance detection circuit in electrical communication with the electrode detection circuit. The fluidic system is formed on the electronic component package and is configured to transport fluid. The electrode array in fluid communication with the fluidic system and includes a plurality of electrode pairs. The electrode array having a surface coating with an antibody. The impedance detection circuit is in electrical communication with the electrode array and is configured to detect an electrical impedance change caused by a binding of the biological sample to the antibody on each of the electrode pairs.

### CROSS-REFERENCE TO RELATED APPLICATIONS

This application is a non-provisional of and claims the benefit of U.S. Provisional Patent Application No. 63/471,375, filed on Jun. 6, 2023, the entire contents of which are incorporated herein by reference.

### BACKGROUND

Millions of people suffer annually from major chronic diseases such as cancers, diabetes, cardiovascular and pulmonary disease, and infectious disease. To improve survival rates of patients and give the right treatment at the right time, early diagnosis of these diseases is required. The most common way to diagnose these diseases is to use an immunoassay which detects various protein biomarkers from blood samples. The current gold standard immunoassay for detecting these biomarkers is enzyme linked immunosorbent assay (ELISA), whereby antibodies in serum samples are tested for binding to antigens specific for a target molecule.

Such assays have significant limitations with respect to the required sample volume, total assay time, expensive fluorescence detector and an inability to multiplex in a single well, with the latter approach resulting in increased costs and reagents. A simpler and more cost-effective approach is still needed to achieve a more efficient immunoassay platform.

A self-contained microfluidic system has attracted broad interest in recent years as an approach for realizing the “lab-on-a-chip” (LOC), or micro total analysis system (μTAS) concept. The objective of this technology is to achieve the integration of sample preparation, purification, and detection in a miniaturized analysis system. In the LOC system, the shorter diffusion distances, convective mass transport and smaller surface-to-volume ratios in microchannels reduce incubation times required for binding reactions. In addition, the ability to integrate multiple fluidic components in a single device eliminates the need for manual sample preparation, prevents contamination between operations, and significantly reduces sample consumption.

Current rapid point of care diagnostic tests are based on lateral flow or “strip-tests,” where a sample fluid moves through a paper strip by capillarity. The presence or absence of a target analyte is indicated by colored small particles that are taken up by the fluid and selectively bind to indicator regions along the strip. This kind of assay is used successfully for high concentration analytes; however, it has proven difficult to extend this technology to lower concentration targets. Current strip tests for low concentration cardiac markers such as CK-MB or troponins all require a reader for detection. A sufficient quantity of analyte is needed to color the detection line and make it visible to the naked eye. Furthermore, the test results can be hard to interpret for unfamiliar users, and there is no quality control or automatic way of collecting test results to quantify disease incidence or facilitate the detection and management of emerging health threats.

Accordingly, it is desirable to provide a low-cost, high-density, and sensitive diagnostic tool that is both user-friendly and automated. Such a device would revolutionize point-of-care testing, particularly in resource-limited settings, where access to sophisticated laboratory equipment is often challenging.

### SUMMARY

The present disclosure provides a novel biosensor that uses complementary metal-oxide-semiconductor (CMOS) based impedance array (IA) technology. This CMOS-IA biosensor has an integrated multimode microelectrode array, a wide bandwidth linear sinusoidal signal synthesizer, and an impedance readout circuit incorporated into a single chip.

Furthermore, a capillary flow-driven fluidic system is incorporated into the sensor chip to perform ultra-low-power immunoassays for point-of-care (POC) diagnostics applications.

The present disclosure also provides a CMOS digital biosensing platform that combines magnetic microparticles with a custom CMOS impedance biosensor array chip for point-of-care testing in resource-challenged environments. The CMOS digital biosensing platform enables efficient data sharing, centralized storage, real-time diagnostics, remote monitoring, and personalized medicine, making it a valuable tool for healthcare, research, and other applications where timely, accurate data is critical.

Microparticles have proven effective as labels for a variety of biomarkers, replacing conventional fluorescent labels in immunoassays. The non-degradable microparticles are insensitive to temperature variations and do not require the sample to be optically transparent. Using the chip for detection enables automated data analysis and collection, taking less than a minute.

The CMOS digital biosensing platform includes a detector chip that is self-contained. The high functionality of modern CMOS technology provides a CMOS chip that performs all functions required to detect and count the microparticles. The CMOS chip uses current conductors embedded in the detector, powered on only when needed, to locally read impedance signals.

The CMOS digital biosensing platform provides robust microparticle detection based on impedance change. The microparticles are superparamagnetic and typically require a large external field for detection. The standard approach of detecting the small (generally less than 1%) increase of the magnetic field in the presence of the microparticles is prone to detection errors caused by temperature changes or stray magnetic fields (e.g., from nearby electrical equipment). In contrast, the CMOS digital biosensing platform disclosed herein measures impedance shift, which is a unique signature from the microparticle that is less sensitive to environmental variations.

In one embodiment, the disclosure provides a point-of-care diagnostic device comprising an electronic component package and a complementary metal-oxide-semiconductor (CMOS) biosensor formed on the electronic component package. The CMOS biosensor includes a fluidic system, an electrode array in fluid communication with the fluidic system, and an impedance detection circuit in electrical communication with the electrode detection circuit. The fluidic system is formed on the electronic component package and is configured to transport fluid. The fluidics system comprises a first inlet configured to receive a buffer fluid, a second inlet configured to receive a biological sample, a reaction channel fluidly connected to the first inlet and the second inlet, wherein the buffer fluid and the biological sample flow through the reaction channel, and a waste bin fluidly connected to the reaction channel. The waste bin configured to receive the buffer fluid and the biological sample. The electrode array in fluid communication with the fluidic system and includes a plurality of electrode pairs. The electrode array configured to perform a multiplexing immunoassay procedure and having a surface coating with an antibody. The impedance detection circuit in electrical communication with the electrode array and is configured to detect an electrical impedance change caused by a binding of the biological sample to the antibody on each of the electrode pairs.

In another embodiment, the disclosure provides a point-of-care diagnostic device comprising an electronic component package and a complementary metal-oxide-semiconductor (CMOS) biosensor formed on the electronic component package. The CMOS biosensor includes a fluidic system, an electrode array in fluid communication with the fluidic system, and an impedance detection circuit in electrical communication with the electrode detection circuit. The fluidic system is formed on the electronic component package and is configured to transport fluid. The fluidic system comprises a first inlet configured to receive a buffer fluid, a second inlet configured to receive a biological sample, a reaction channel fluidly connected to the first inlet and the second inlet, wherein the buffer fluid and the biological sample travel through the reaction channel, and a waste bin fluidly connected to the reaction channel. The waste bin configured to receive the buffer fluid and the biological sample. The electrode array configured to perform a multiplexing immunoassay procedure. The electrode array in fluid communication with the fluidic system and comprises a first electrode array section having a surface coating with a first antibody, a second electrode array section having a surface coating with a second antibody, a third electrode array section having a surface coating with a third antibody, and a fourth electrode array section having a surface coating with a fourth antibody. The impedance detection circuit in electrical communication with the electrode array and is configured to detect an electrical impedance change caused by a binding of the biological sample to the antibody on each of the electrode pairs.

In a further embodiment, the disclosure provides a point-of-care diagnostic device comprising an electronic component package and a complementary metal-oxide-semiconductor (CMOS) biosensor formed on the electronic component package. The CMOS biosensor includes a fluidic system, an electrode array in fluid communication with the fluidic system, an impedance detection circuit in electrical communication with the electrode detection circuit, and impedance-to-digital converter in electrical communication with the impedance detection circuit. The fluidic system is formed on the electronic component package and is configured to transport fluid. The fluidic system comprises a first inlet configured to receive a buffer fluid, a second inlet configured to receive a biological sample, a reaction channel fluidly connected to the first inlet and the second inlet, wherein the buffer fluid and the biological sample travel through the reaction channel, and a waste bin fluidly connected to the reaction channel. The waste bin configured to receive the mixed buffer fluid and the biological sample. The electrode array in fluid communication with the fluidic system and includes a plurality of electrode pairs. The electrode array configured to perform a multiplexing immunoassay procedure and having a surface coating with an antibody. The impedance detection circuit in electrical communication with the electrode array and is configured to detect an electrical impedance change caused by a binding of the biological sample to the antibody on each of the electrode pairs. The impedance-to-digital converter is configured to convert a magnitude and a phase of impedance to a digital signal.

Other aspects of the invention will become apparent by consideration of the detailed description and accompanying drawings.

### BRIEF DESCRIPTION OF THE DRAWINGS

FIG. 1A is a schematic perspective view of a point-of-care device according to an embodiment of the present disclosure.

FIG. 1B is a perspective view of an exemplary point-of-care device of FIG. 1A compared to U.S. one cent coin.

FIG. 2A is a schematic view of fluidic system of the point-of-care device of FIG. 1A.

FIG. 2B is a schematic view of a second inlet of the fluidic system of FIG. 2A.

FIG. 2C is a close-up schematic view of the second inlet, a sample channel, a first capillary valve, and a portion of the reaction channel of the fluidic system of FIG. 2A.

FIG. 3A is a schematic view of the fluidic system of FIG. 1A in a first configuration.

FIG. 3B is a schematic view of the fluidic system of FIG. 1A in a second configuration.

FIG. 3C is a schematic view of the fluidic system of FIG. 1A in a third configuration.

FIG. 3D is a schematic view of the fluidic system of FIG. 1A in a fourth configuration.

FIG. 4 is a schematic section view along line 4-4 of the sensor chip of the point-of-care device of FIG. 1A.

FIG. 5 is a schematic top view of the sensor chip of the point-of-care device of FIG. 1A.

FIG. 6 is a schematic view of the electrode array and an impedance detection circuit of the sensor chip of FIG. 5.

FIG. 7 is another schematic view of the electrode array of FIG. 6.

FIG. 8A is a close-up schematic view of a plurality of working electrodes and a plurality of counter electrodes of the electrode array of FIG. 6.

FIG. 8B is a close-up schematic view of another embodiment of a plurality of working electrodes and a plurality of counter electrodes of FIG. 8A.

FIG. 8C is a close-up schematic view of another embodiment of a plurality of working electrodes and a plurality of counter electrodes of FIG. 8A.

FIG. 9A is schematic section view of the sensor chip of FIG. 5 showing the working electrode, the counter electrode, and a plurality of detection antibodies.

FIG. 9B is a schematic cross-section of an electrode from the electrode array of FIG. 6.

FIG. 10A is a schematic view of a clock signal generator of the impedance detection circuit converter of FIG. 6.

FIG. 10B is a schematic diagram of an exemplary relaxation oscillator of the clock signal generator of FIG. 10A.

FIG. 10C is a schematic diagram of an exemplary injection locked frequency multiplier (ILFM) of the clock signal generator of FIG. 10A.

FIG. 11 is a schematic diagram of a sinusoidal signal generator of the impedance detection circuit of FIG. 6.

FIG. 12 is a schematic diagram of the working electrode, a microparticle counter electrode, and a reference counter electrode directing an output current through a set of transimpedance amplifier.

FIG. 13 is a schematic diagram of an impedance-to-digital converter of the impedance detection circuit converter of FIG. 6.

FIG. 14A is a schematic diagram of a magnitude detection circuit of the impedance-to-digital converter of FIG. 13.

FIG. 14B is a graphical representation of an input and an output of the magnitude detection circuit of the impedance-to-digital converter of FIG. 13.

FIG. 15A is a schematic diagram of a phase detection circuit of the impedance-to-digital converter of FIG. 13.

FIG. 15B is a graphical representation of an input and an output of the phase detection circuit of the impedance-to-digital converter of FIG. 13.

FIG. 16A is perspective view of a plasma separation membrane testing device.

FIG. 16B is a graph comparing the results of UV-V spectroscopy on plasma separated using the plasma separation membrane and plasma separated by a centrifuge.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

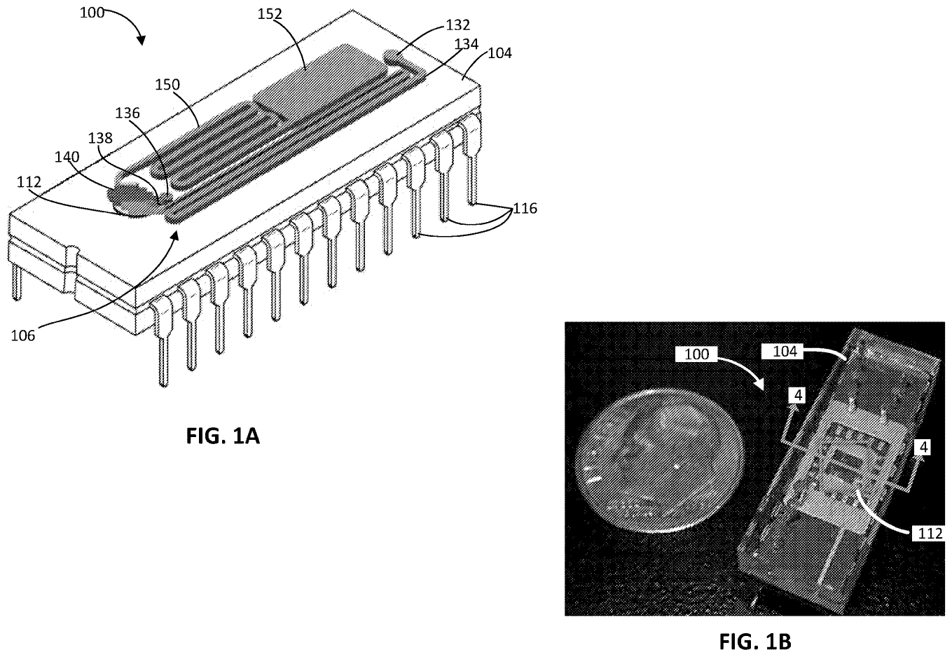

FIG. 1A illustrates a schematic perspective view of a point-of-care device 100, and FIG. 1B illustrates another perspective view of the point-of-care device 100. The point-of-care device 100 is configured to detect the presence of biomarkers (e.g., cardiac biomarkers CK-MB, Myoglobin, cTnl, and NT-proBNP) in a blood sample and is formed on an electronics package 104. In the illustrated embodiment, the electronic package 104 is a ceramic dual inline package (C-DIP) including 48 pins 116 with an overall footprint of for example, 50.8 mm by 15.5 mm or 2 inches by 0.61 inches. In other embodiments, the electronic package 104 is a flexible circuit board including at least 40 pins 116. In further embodiments, the electronic package 104 may include more or less than 40 pins and may have an overall footprint larger or smaller than 50.8 mm by 15.5 mm. Additionally, the electronic package 104 comprises a complementary metal-oxide semiconductor (CMOS) biosensor 106. In some embodiments, the CMOS biosensor 106 is configured to use at most 2 mW of power. In other embodiments, the CMOS biosensor 106 may use more or less than 2 mW of power. The CMOS biosensor 106 comprises a fluidic system 108 and a sensor chip 112 positioned in a chip cavity 114.

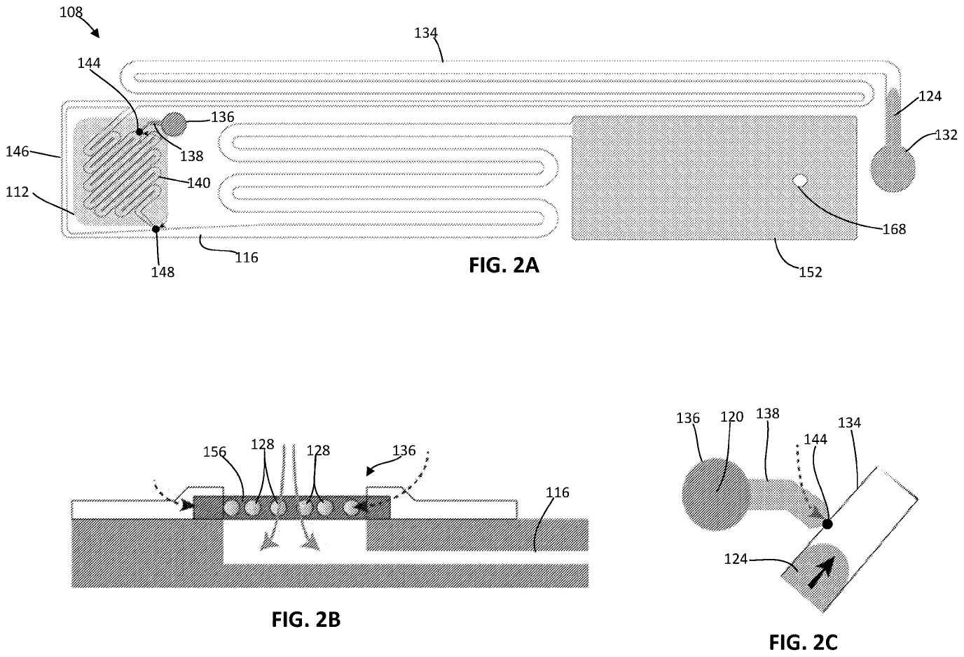

FIGS. 2A-C illustrate different schematic views of the fluidic system 108. The fluidic system 108 is configured to intake and transport the biological sample 120 (e.g., blood), a buffer fluid 124, and a plurality of microparticles 128 along a top surface of the electronics package 104. In the illustrated embodiment, the fluidic system 108 is a self-contained capillary microfluidic system and comprises a first inlet 132, a buffer channel 134, a second inlet 136, a sample channel 138, a reaction channel 140, a first capillary valve 144, a buffer detour channel 146, a second capillary valve 148, a waste channel 150, and a waste bin 152.

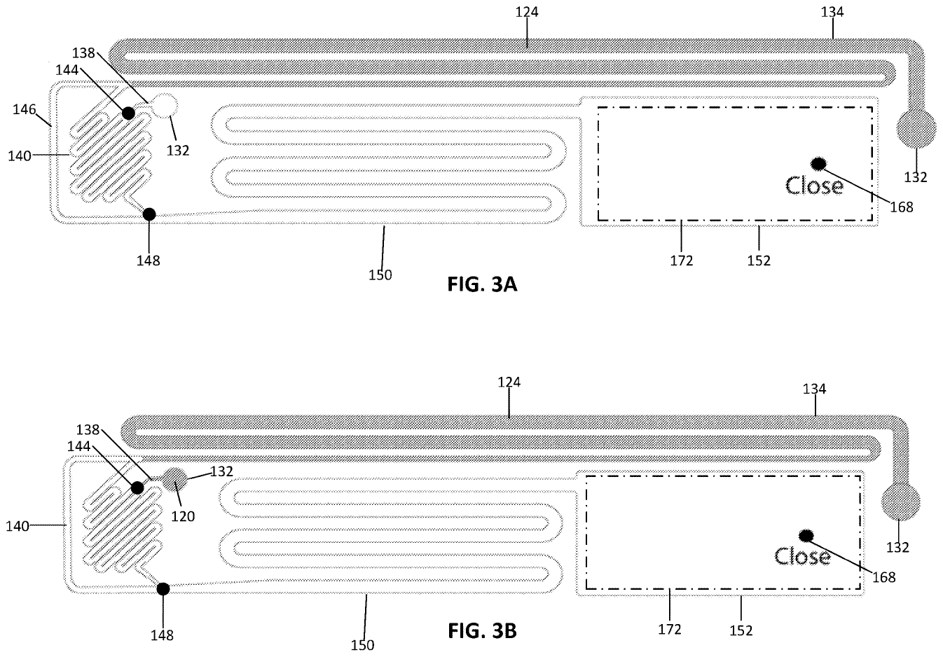

With continued reference to FIGS. 2A-C, the first inlet 132, the buffer channel 134, and buffer detour channel 146 are shown. The first inlet 132 is configured to receive the buffer fluid 124 (e.g., phosphate buffer saline (PBS) or Bovine Serum Albumin (BSA)). The buffer channel 134 fluidly connects the first inlet 132 to the first capillary valve 144. The buffer detour channel 146 fluidly connects the 132 to the second capillary valve 148. In the illustrated embodiment, both the buffer channel 134 and the buffer detour channel 146 include a wicking matrix (not shown), which allows the buffer fluid 124 to travel through the buffer channel 134 and buffer detour channel 146 by capillary action, as shown in FIG. 3A. In other embodiments, the buffer fluid 124 may travel through the buffer channel 134 or buffer detour channel 146 due to a pressure differential or gravity driven flow.

FIGS. 2A-C illustrate the second inlet 136 configured to receive the biological sample 120 (e.g., blood). The second inlet 136 comprises a plasma separation membrane 156, as shown in FIG. 2B. The plasma separation membrane 156 is a porous membrane embedded in the second inlet 136 and separates the surrounding environment from the sample channel 138. Additionally, the plasma separation membrane 156 is configured to house the plurality of microparticles 128, conjugated with a plurality of detection antibodies 160. When the biological sample 120 travels through the plasma separation membrane 156, plasma is separated from the biological sample 120 and the detection antibodies 160 of the microparticles 128 bind to target molecules 164 in the plasma. In the illustrated embodiment, the plasma separation membrane 156 is a GX membrane from Pall Corp, but in other embodiments the plasma separation membrane 156 may be a different type of porous membrane. In further embodiments, the second inlet 136 may include two porous membranes, where one membrane is only configured to separate the plasma from the blood and another membrane is configured to house the microparticles 128.

As shown in FIG. 2A-C, the microparticles 128 are spherical magnetic beads with a diameter of 2.8 μm and are conjugated with a plurality of detection antibodies 160. In other embodiments, the microparticles 128 may be shaped differently and may have a diameter larger than 2.8 μm. To couple the microparticles 128 to the plasma separation membrane 156, the microparticles 128 are first deposited on the plasma separation membrane 156 and then a layer of Polydimethylsiloxane (PDMS) is applied over the plasma separation membrane 156. After curing at room temperature, the PDMS creates a bond between the microparticles 128 and the plasma separation membrane 156.

As shown in FIGS. 2A-C, the sample channel 138 is in fluid communication with the second inlet 136 and is selectively in fluid communication with the remainder of the fluidic system 108 through the first capillary valve 144. Additionally, the sample channel 138 includes a wicking matrix (not shown), which allows the biological sample 120 to travel through the sample channel 138. In other embodiments, the biological sample 120 may travel through the sample channel 138 due to a pressure differential or a gravity driven flow.

As shown in FIGS. 2A-C, the first capillary valve 144 separates the buffer channel 134 and the sample channel 138 from the reaction channel 140. The first capillary valve 144 is configured to regulate fluid flow based on the alteration of surface properties or geometries. In the illustrated embodiment, the first capillary valve 144 is configurable between an open state, in which fluid flows freely, and a closed state in which fluid flow is prevented. The open state of the first capillary valve 144 occurs when both biological sample 120 and buffer fluid 124 are in contact with the first capillary valve 144. Otherwise, the first capillary valve 144 will be in the closed state. Once in the open state, the biological sample 120 enters the reaction channel 140 followed by the buffer fluid 124.

As shown in FIGS. 2A-C, the reaction channel 140 begins after the first capillary valve 144 and ends at the second capillary valve 148. The reaction channel 140 is a portion of the fluidic system 108 where the biological sample 120 and the buffer fluid 124 pass over the sensor chip 112 for a desired reaction time. The desired reaction time is based on the length of the reaction channel 140, and in other embodiments the length may be increased to increase the desired reaction time and vice versa.

As shown in FIGS. 2A-C, the second capillary valve 148 separates the reaction channel 140 and the buffer detour channel 146 from the waste channel 150. Like the first capillary valve 144, the second capillary valve 148 is configurable between an open state, in which fluid flows freely, and a closed state in which fluid flow is prevented. In the illustrated embodiment, the second capillary valve 148 is in an open state when both the biological sample 120 in the reaction channel 140 and the buffer fluid 124 in the buffer detour channel 146 reach the second capillary valve 148. Once in the open state, the biological sample 120 and buffer fluid 124 enter the waste channel 150, which includes a wicking matrix (not shown). The biological sample 120 and buffer fluid 124 travel through the waste channel 150 and empty into the waste bin 152. The flow through the waste channel 150 is a result of capillary action through the wicking matrix and a pressure differential between the waste bin 152 and the remainder of the fluidic system 108.

As shown in FIGS. 2A-C, the waste bin 152 is positioned at the end of the waste channel 150 and receives the capillary flow of the biological sample 120 and buffer fluid 124 from the waste channel 150. Once the biological sample 120 and the buffer fluid 124 reach the waste bin 152, the capillary flow is slowed significantly. In the illustrated embodiment, the waste bin 152 includes an outlet vent 168 and an absorbent pad 172. The outlet vent 168 is configurable between an open position, when the first capillary valve 144 is in the open position, and a closed position, when the first capillary valve 144 is in the closed position. When the outlet vent 168 is in an open position (FIG. 3C), the waste bin 152 is fluidly connected to the surrounding environment and creates a pressure differential promoting the flow of the mixture of biological sample 120 and buffer fluid 124 through the reaction channel 140. Additionally, the outlet vent 168 allows the user to drain the accumulated biological sample 120 and the accumulated buffer fluid 124. The absorbent pad 172 absorbs the biological sample 120 and buffer fluid 124 and maintains an overall flow rate for effective hydrodynamic washing.

FIGS. 3A-D illustrate the configurations of the fluidic system 108 during operation. As shown in FIG. 3A, the user starts by loading the buffer fluid 124 into the first inlet 132. The buffer fluid 124 flows through the buffer channel 134 towards the first capillary valve 144.

Next, as shown in FIG. 3B, the user deposits blood (the biological sample 120) into the second inlet 136 and the plasma separation membrane 156 separates the plasma from the deposited blood. While traveling through the plasma separation membrane 156, the blood interacts with the plurality of microparticles 128, and the detection antibodies 160 of the microparticles 128 selectively bind to the target molecules 164 (e.g., biomarker) in the separated plasma. Afterwards, the separated plasma and a number of microparticles 128 bound to target molecules 164 enter the sample channel 138.

Then, as shown in FIG. 3C, the first capillary valve 144 is transitioned into the open state, when reached by both the biological sample 120 and buffer fluid 124. The biological sample 120 begins to flow into the reaction channel 140 followed by the buffer fluid 124. As the biological sample 120 flows through the reaction channel 140, the target molecules 164 of the biological sample 120 react with the sensor chip 112, and a number of the target molecules 164 bind to the surface of the sensor chip 112, as described in greater detail below. Additionally, the microparticles 128 bounded to the target molecules 164 assist in even distribution of the target molecules 164 across the entire sensor chip 112. Simultaneously, the buffer fluid 124 enters the buffer detour channel 146 and the outlet vent 168 of the waste bin 152 is opened.

Finally, as shown in FIG. 3D, the biological sample 120 and the buffer fluid 124 from the buffer detour channel 146 contact the second capillary valve 148 to transition the second capillary valve 148 into the open state. As a result, both the biological sample 120 and the buffer fluid 124 enter the waste channel 150 and flow towards the waste bin 152. Additionally, as the biological sample 120 is emptied from the reaction channel 140, the remainder of the buffer fluid 124 in the reaction channel 140 collects microparticles 128 accumulated on the surface of the sensor chip 112 and follows the biological sample 120 into the waste channel 150.

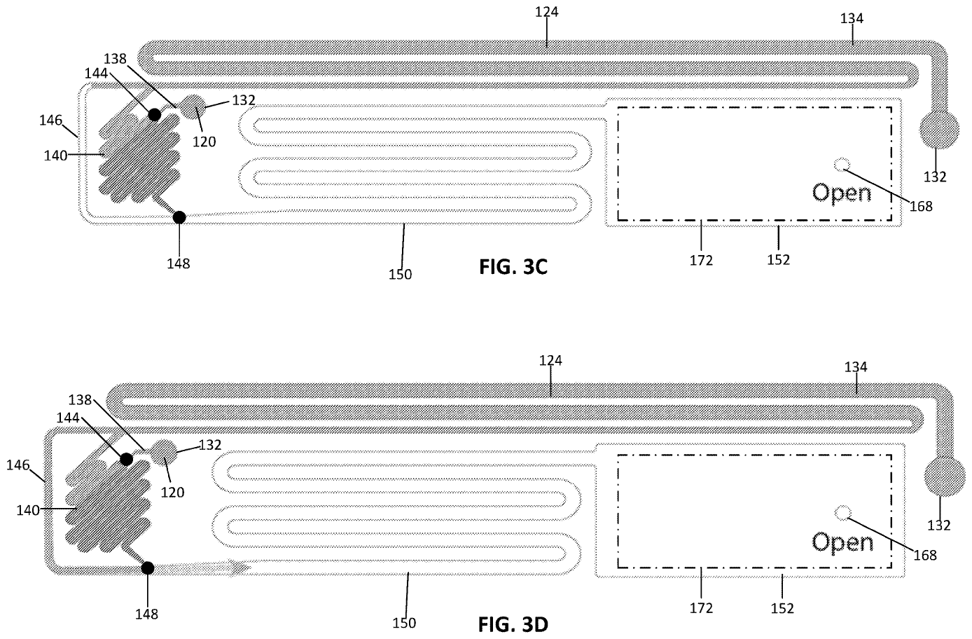

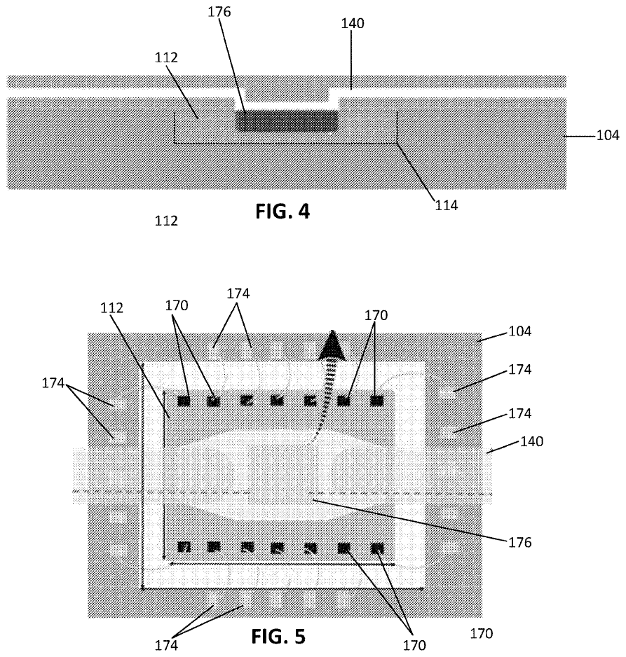

As shown in FIGS. 4-6, the sensor chip 112 is positioned in a chip cavity 114 in the electronics package 104. The sensor chip 112 is configured to perform a multiplexing immunoassay procedure to measure at least one characteristics of the biological sample 120 and send at least one signal, representing the characteristics of the biological sample 120, through the pins 116 of the electronics package 104. The sensor chip 112 is electrically connected to the electronics package 104 and is fluidly connected with at least a portion of the reaction channel 140. The electrical connection between the sensor chip 112 and the electronics package 104 is formed through electrically connecting a plurality of pads 170 to a plurality of leads 174. In the illustrated embodiment, the plurality of pads 170 include at least one pad for receiving an input voltage, at least one pad for electrode selection, and at least one pad for reading current. The fluid connection between the reaction channel 140 and the sensor chip 112 allows the mixture of the biological sample 120 and the buffer fluid 124 to flow over the sensor chip 112. In the illustrated embodiment, the sensor chip 112 is manufactured on a 180nm semiconductor manufacturing process, but in other embodiments, the sensor chip 112 may be manufactured with a different semiconductor manufacturing process. The sensor chip 112 includes an electrode array 176, and an impedance detection circuit 200.

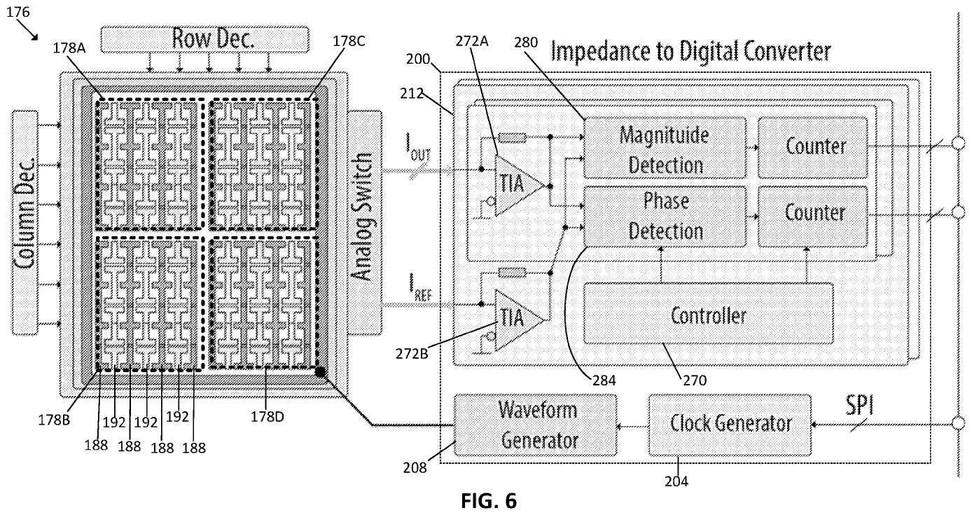

FIGS. 6, 7, and 8A illustrate an electrode array 176. In the illustrated embodiment, the electrode array 176 is positioned centrally on the sensor chip 112 and is in fluid communication with the reaction channel 140. The electrode array 176 comprises a plurality of working electrodes 188, a plurality of counter electrodes 192, and a plurality of capture antibodies 196 positioned in between the working and counter electrodes 188, 192. Additionally, the electrode array 176 is split into four electrode subarrays 178A, 178B, 178C, 178D each including 16,384 working electrodes 188 and counter electrodes 192 arranged in a grid of 128 rows by 128 columns. In other embodiments, the electrode array 176 may include more or less than 4 electrode subarrays and each electrode subarray may of a different dimension.

As shown in FIGS. 6, 7, 8A, and 9A-B, the working electrodes 188 and the counter electrodes 192 are arranged in pairs on a top surface of the sensor chip 112. In the illustrated embodiment, the working electrodes 188 are cross-shaped and the counter electrodes 192 are T-shaped. As a result, the shapes of the working electrodes 188 and the counter electrodes 192 are complementary to one another and can be arranged with reduced spacing. Additionally, in the illustrated embodiment, the working and counter electrodes 188, 192 are at most 5 μm spaced apart from one another. In other embodiments, the spacing between the working and counter electrodes 188, 192 may be larger or smaller than 5 μm to achieve different degrees of sensitivity.

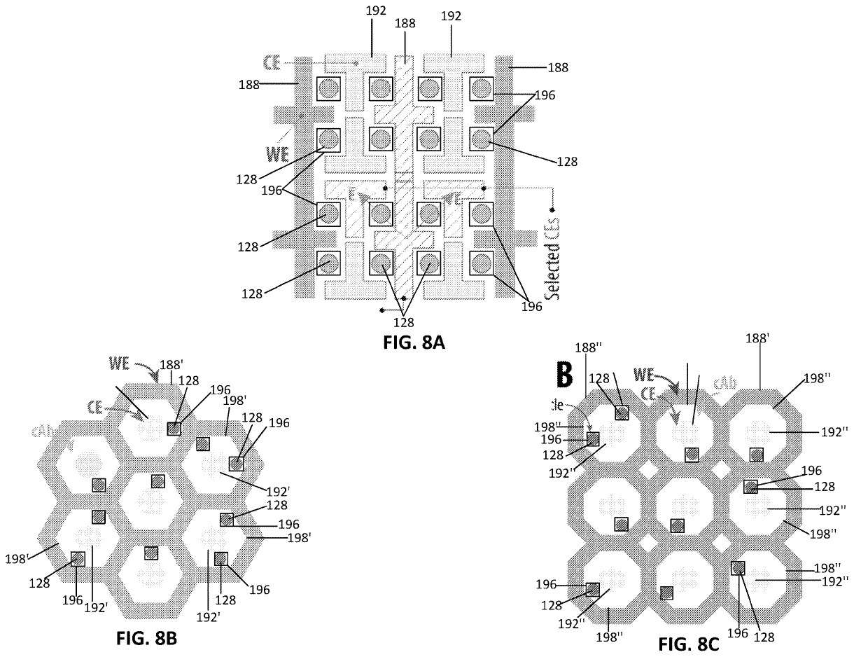

FIG. 8B illustrates another embodiment of a plurality of working electrodes 188′ and a plurality of counter electrodes 192′, with like parts to the working electrodes 188 and the counter electrodes 192, respectively. The working electrode 188′ is hexagonal-shaped including a centrally positioned hexagonal opening 198′. The counter electrode 192′ is also hexagonal-shaped and is positioned within the hexagonal opening 198′ of the working electrode 188′. Additionally, the working electrodes 188′ are arranged in a honeycomb structure. The hexagonal shape of the working and counter electrodes 188′, 192′ establish a more constant electric field and improved area efficiency, compared to alternatively shaped working and counter electrodes 188′, 192′.

FIG. 8C illustrates another embodiment of a plurality of working electrodes 188″ and a plurality of counter electrodes 192″, with like parts to the working electrodes 188″ and the counter electrodes 192″. The working electrodes 188″ has an octagonally shaped cross section including a centrally positioned octagonal opening 198″. The counter electrodes 192″ also have an octagonally-shaped cross-section and is positioned within the octagonal opening 198″ of the working electrodes 188″. Additionally, the working electrodes 188″ are arranged in a grid pattern. The octagonal shape of the working and counter electrodes 188″, 192″ allow for easy alignment of a sensing circuit (not shown), because both the X and Y direction may be accessed in a straight line.

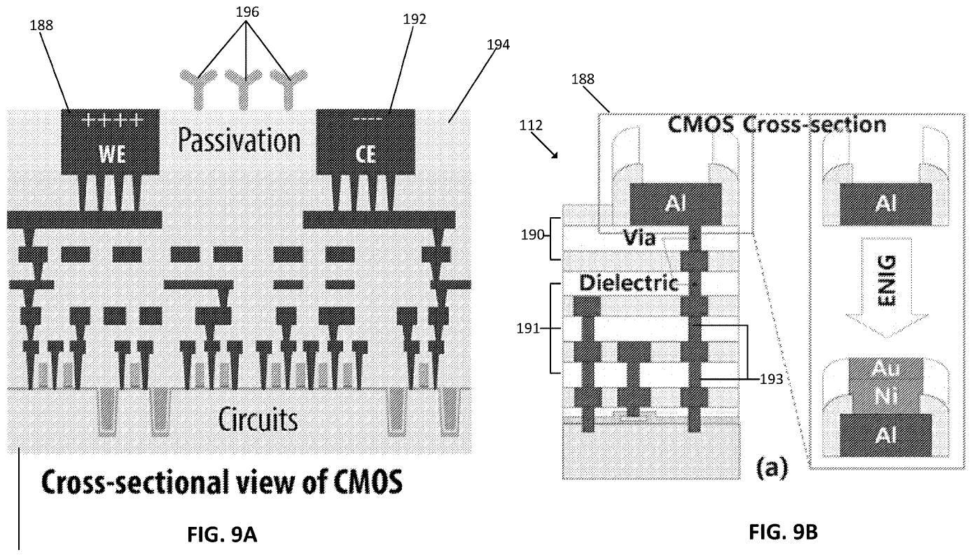

FIGS. 9A and 9B illustrate a cross-section of the sensor chip 112 focusing on the working and counter electrodes 188, 192. FIG. 9A shows a plurality of electrical connections extending from the working and counter electrodes 188, 192 through a plurality of conductive layers 190 and a plurality of dielectric layers 191 of the sensor chip 112 using a plurality of vias 193. FIG. 9B shows that each of the working and counter electrodes 188, 188′, 188″, 192, 192′, 192″ are composed of aluminum and are formed through a complementary metal-oxide-semiconductor (CMOS) fabrication process. Afterwards, the working and counter electrodes 188, 192 receive an inert electrode layer 198 composed of an inert metallic material (e.g., gold or platinum). In some embodiments, the inert electrode layer 198 is gold and is applied using electroless nickel immersion gold (ENIG) process. The gold plating through the ENIG process reduces the chance of an undesired short between the working electrodes 188 and the counter electrodes 192 due to contact with one of the microparticles 128. Finally, a passivation process deposits a passivation layer 194 (e.g., a thin layer of a chemically inert, corrosion-resistant, dielectric material) filling the space between the working and counter electrodes 188, 192, as shown in FIG. 9A.

In operation, the working electrode 188 is activated to apply an electric field to an adjacent counter electrode 192 to measure the impedance of the biological sample 120. However, not all working electrodes 188 apply an electric field to the adjacent counter electrode 192 at all times during operation. Instead, a sliding access operational method selectively activates groups of working electrodes 188 until each working electrode 188 of the electrode array 176 is activated. By not simultaneously activating working electrode 188, interference is reduced.

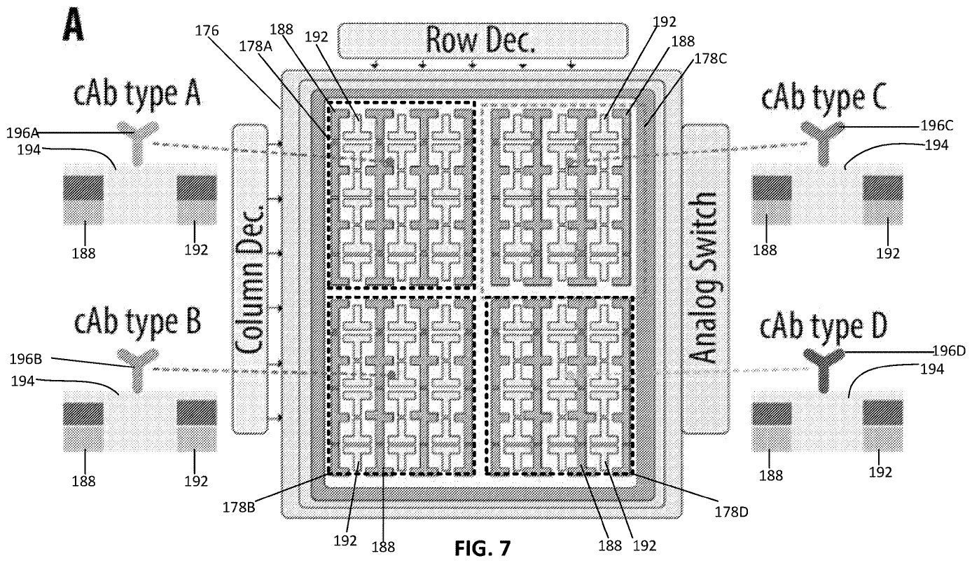

As shown in FIGS. 7 and 9A, the capture antibodies 196 are positioned in between the working and counter electrodes 188, 192 on the passivation layer 194. In the illustrated embodiment, the sensor chip 112 includes four types of captures antibodies 196A, 196B, 196C, 196D, and each type of capture antibodies 196A, 196B, 196C, 196D correspond to one of the electrode subarrays 178A 178B, 178C, 178D. Additionally, each type of capture antibodies 196A, 196B, 196C, 196D are configured to bind with a different target molecule 164 (e.g., cardiac biomarkers CK-MB, Myoglobin, cTnl, and NT-proBNP). In other embodiments, the sensor chip 112 may include more or less than four distinct types of capture antibodies 196. By including multiple types of capture antibodies 196, the sensor chip 112 is configured to simultaneously the presence of different target molecules 164.

To manufacture the sensor chip 112, the sensor chip 112 is first placed the chip cavity 114 in the electronics package 104. Next, the pads 170 of the sensor chip 112 are electrically connected to the leads 174 of the electronics package 104 through wire bonding. Then, the sensor chip 112 is covered in a microfabrication-grade electrostatic film (e.g., Rubylith, 3M) to protect the surface of the sensor chip 112. Afterwards, the chip cavity 114 is filled with polydimethylsiloxane (PDMS) to planarize the top surface of the electronic package 104. Next, the electrostatic film is removed to expose the top surface of the sensor chip 112. Then, the top surface of the sensor chip 112 is activated using 3-Aminopropyltriethoxysilane (APTES) to form an amine group through a condensation reaction. Afterward, the top surface of the sensor chip 112 is washed and the capture antibodies 196 are coupled to the electrode array 176 through carbodiimide couple chemistry. Lastly, the fluidic system 108 is bonded to the sensor chip 112 after an oxygen plasma treatment.

The impedance detection circuit 200 is configured to measure the impedance of a material between the working electrodes 188 and the counter electrodes 192 and output a digital signal representing the impedance. In the illustrated embodiment, the impedance detection circuit 200 is a wideband impedance readout circuit powered by at most 0.8 mW. Additionally, in the illustrated embodiment, the impedance detection circuit 200 is a polar demodulator. In other embodiments, the impedance detection circuit 200 may also be an in-phase/quadrature-phase demodulator. The impedance detection circuit 200 comprises a clock signal generator 204, a sinusoidal signal generator 208, and an impedance-to-digital converter (IDC) 212.

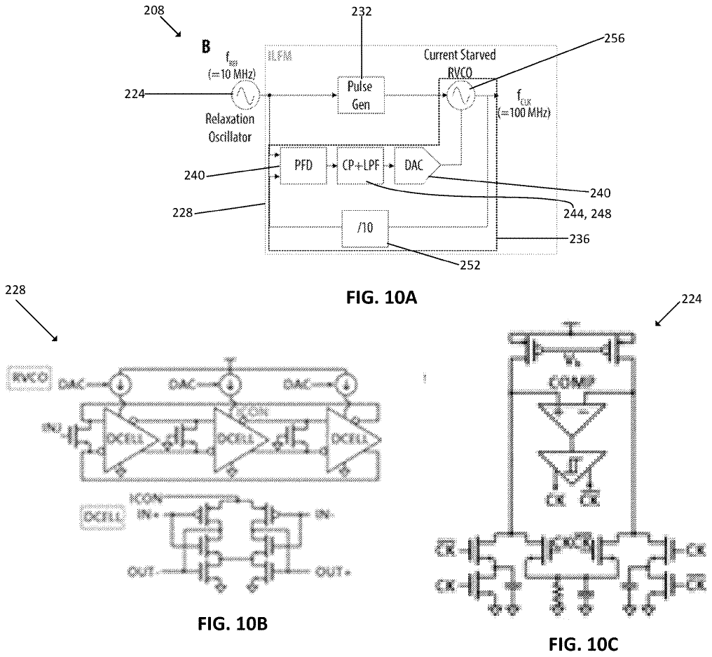

FIGS. 10A-C illustrate the clock signal generator 204, which is configured to output a clock frequency (f_{clk}) of 100 Mhz. In other embodiments, the f_{clk} may be greater or less than 100 Mhz. The clock signal generator 204 includes a relaxation oscillator 224 (FIG. 10B), and an injection-locked frequency multiplier (ILFM) 228 (FIG. 10C). The relaxation oscillator 224 is formed on the sensor chip 112 and receives power in the range of tens of picowatts to output a reference clock frequency (f_{ref}) in the range of tens of Mhz. In the illustrated embodiment, reference clock frequency is 10 Mhz, but in other embodiments, the reference clock frequency may be greater or less than 10 Mhz. The reference clock frequency (f_{ref}) becomes an input for the ILFM 228.

As shown in FIGS. 10A and 10C, the ILFM 228 is configured to multiply the reference clock frequency (f_{ref}) by a set multiplier and output a clock frequency (f_{clk}) in the range of several hundred Mhz with minimal power consumption. In the illustrated embodiment, the clock frequency (f_{clk}) is 100 Mhz, but in other embodiments, the clock frequency (f_{clk}) may be greater or less than 100 Mhz. Additionally, in the illustrated embodiment, ILFM 228 includes a pulse generator 232 and a phase locked loop 236. The pulse generator 232 generates rectangular pulses and is connected in parallel to the phase locked loop 236. The phase locked loop 236 comprises a phase frequency detector (PFD) 240, a charge pump (CP) 244, a low pass filter (LPF) 248, a digital to analog converter (DAC) 252, and a ring voltage-controlled oscillator (RVCO) 256. The PFD 240 compares the phases of f_{ref} from the relaxation oscillator 224 and a scaled clock frequency, and outputs a corresponding error voltage. The scaled clock frequency is based on a divider 252, which is equal to 10 in the illustrated embodiment. The charge pump 244 and the low-pass filter 248 are configured to output a tuning voltage for the RVCO 256, based on the corresponding error voltage from the PFD 240. The RVCO 256 will then adjust a frequency multiplier applied to an output of the pulse generator 232 to reach the desired clock frequency. Additionally, the RVCO 256 is configured to limit the phase noise performance. In other embodiments, a frequency locked loop (FLL) may be connected in parallel with the ILFM 228 to reduce power consumption when the ILFM 228 is locked.

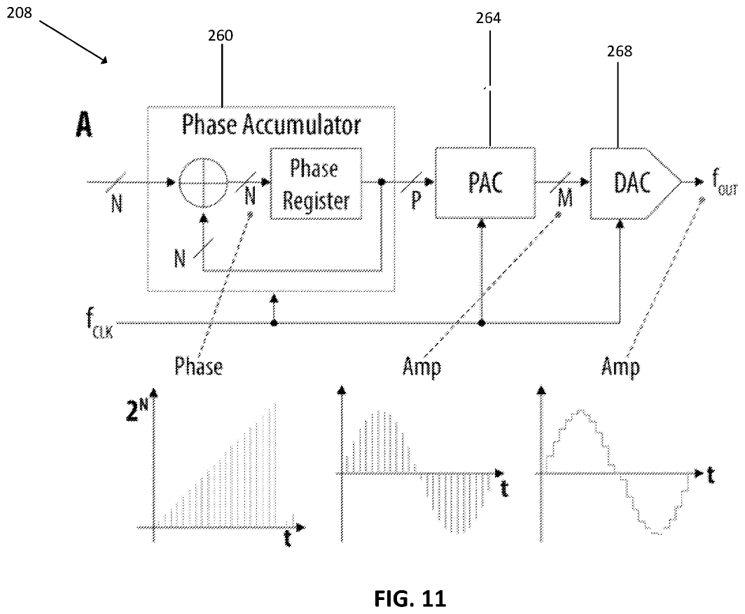

FIG. 11 illustrates the sinusoidal signal generator 208 configured to generate an analog sinusoidal output signal (fₒᵤₜ) using a turning word (“N”) and the clock signal (f_{clk}). In the illustrated embodiment, the sinusoidal signal generator 208 is a digital to analog converter based direct digital synthesizer (DDS) with high order harmonics remover. Additionally, the illustrated sinusoidal signal generator 208 is wideband and powered using less than 1.2 mW. The sinusoidal signal generator 208 comprises a phase accumulator 260, a phase to amplitude converter (PAC) 264, and a digital to analog converter (DAC) 268. The turning word “N” is optimized by the user to achieve a desired frequency of the sinusoidal output signal (fₒᵤₜ). The clock signal (f_{clk}) is received from the clock signal generator 204 and is received by the phase accumulator 260, the phase to amplitude converter 264, and the digital to analog converter 268. The phase accumulator 260 is configured generate a plurality of digital states, where each consecutive digital state increases linearly. The PAC 264 receives the plurality of digital states from the phase accumulator 260 and converts the digital states into a digital sine wave. Finally, the DAC 268 receives the digital sine wave from the phase accumulator 260 and converts the digital sinewave into an analog sinusoidal output signal (fₒᵤₜ).

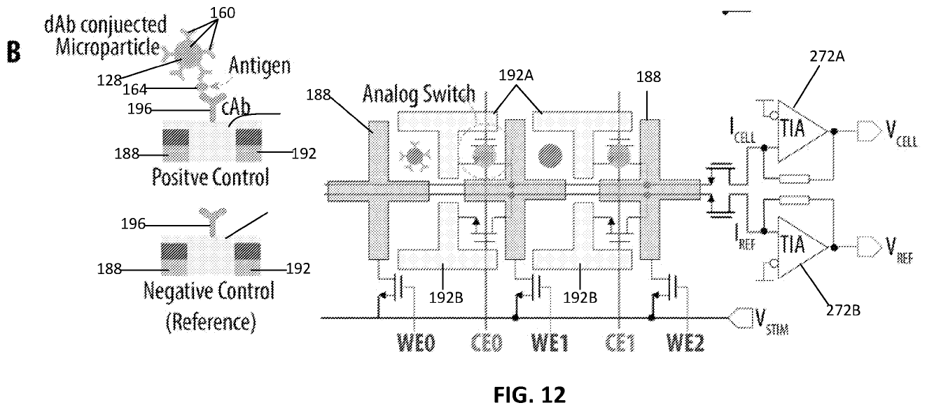

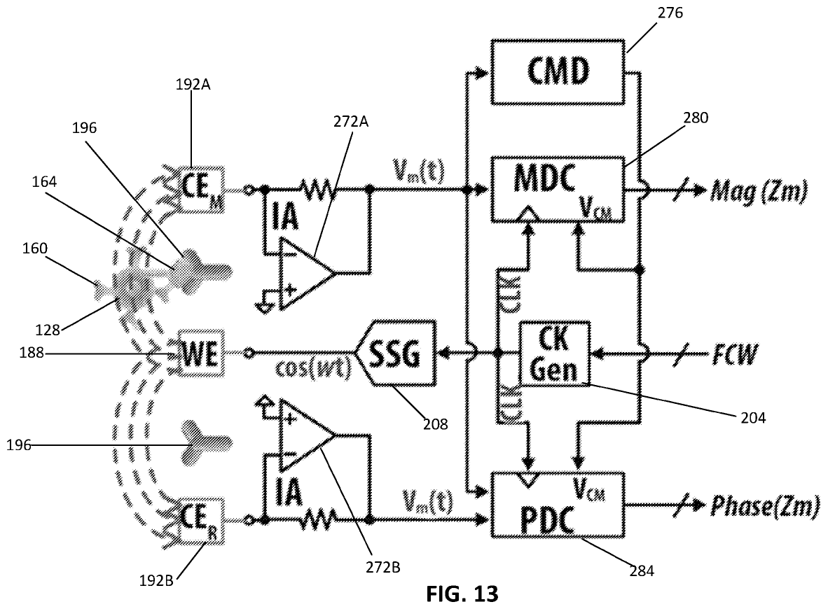

As shown in FIGS. 12 and 13, a current proportional to the analog sinusoidal output signal (fₒᵤₜ) is applied to the working electrode 188. The current passes through the biological sample 120 and is received by both a microparticle counter electrode 192A and a reference counter electrodes 192B. The current received by the microparticle electrode (I_{cell}), and the current received by the reference electrode (I_{ref}) is passed through a microparticle transimpedance amplifier 272A and a reference transimpedance amplifier 272B, respectively.

Both the microparticle and reference transimpedance amplifiers 272A, 272B are configured to convert the current received from the microparticle and reference counter electrodes 192A, 192B into a microparticle voltage Vₘ and a reference voltage Vᵣ, respectively.

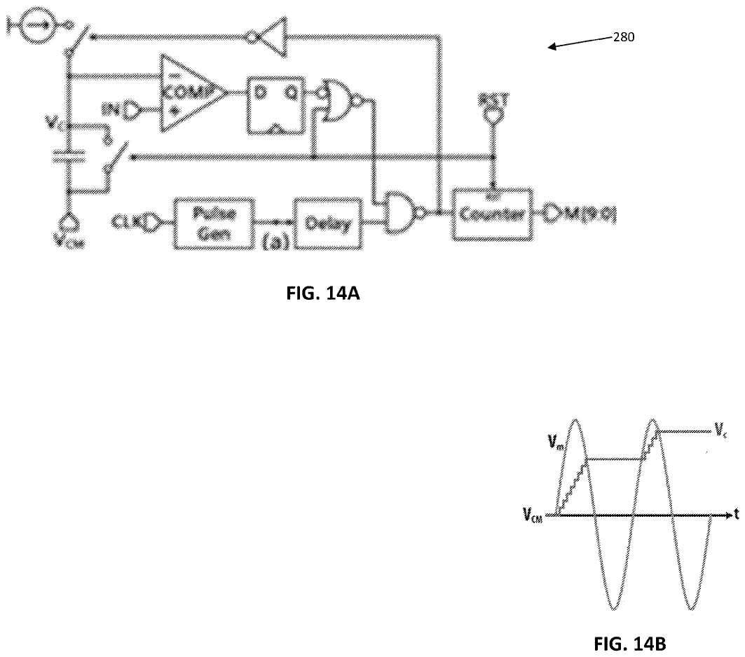

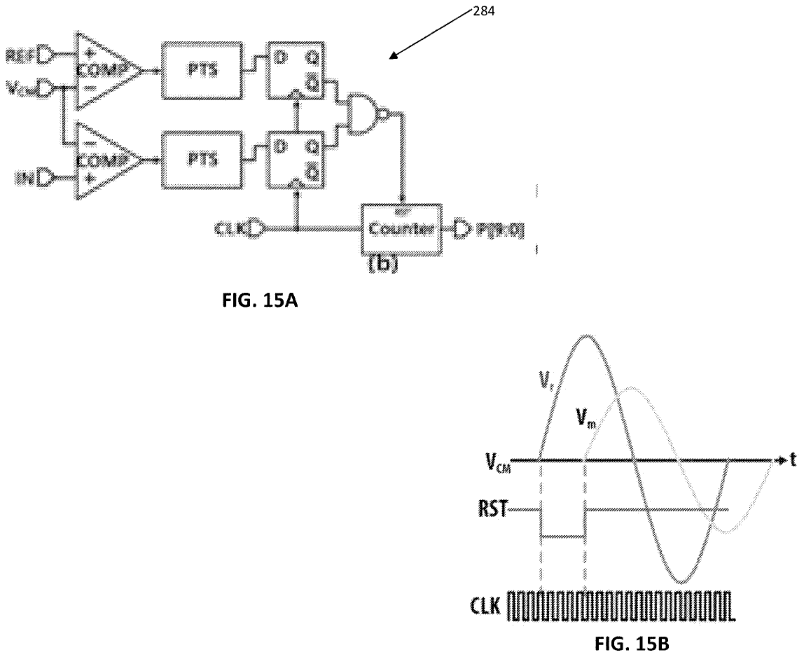

As shown in FIGS. 6 and 13, the output of the microparticle and reference transimpedance amplifiers 272A, 272B are electrically coupled to the impedance-to-digital converter (IDC) 212. The IDC 212 comprises a controller 270, a common mode detector (CMD) 276, a magnitude-to-digital converter (MDC) 280 (FIG. 14A), and a phase-to-digital converter (PDC) 284 (FIG. 15A). The CMD 276 is configured to receive the microparticle voltage Vₘ and output a common mode level V_{cm}. The MDC 280 is configured to receive the microparticle voltage Vₘ, the common mode level V_{cm}, and the clock signal (f_{clk}). In operation, the MDC 280 compares the amplitude of the microparticle voltage Vₘ to the common mode level V_{cm} and determines a magnitude V_{c}. As shown in FIG. 14B, the magnitude V_{c} is equivalent to the peak of the microparticle voltage Vₘ and is calculated by incrementally increasing the magnitude V_{c} in sync with the clock signal (f_{clk}), until magnitude V_{c} matches the peak of the microparticle voltage Vₘ. The PDC 284 receives the reference voltage Vᵣ, the microparticle voltage Vₘ, the common mode level V_{cm}, and the clock signal (f_{clk}). In operation, the PDC 284 generates an RST signal constructed based on the relationship between the reference voltage (Vᵣ), the microparticle voltage (Vₘ), and the common mode level (V_{cm}). Specifically, the falling edge of the reset signal (RST) is determined when Vᵣ intersects with V_{cm} and the rising edge is determined when (Vₘ) crosses the (V_{cm}) peak, as shown in FIG. 15B. To calculate phase, the PDC 284 counts the number of clock cycles during the RST signal alteration. With the phase from the PDC 284 and the magnitude V_{c} from the MDC 280, the controller 270 can determine impedance of the biological sample 120 between the working electrode 188 and the counter electrode 192.

In operation, the biological sample 120 flows past the working and counter electrodes 188, 192 of the sensor chips 112, as shown in FIG. 3C. When the biological sample 120 is in proximity to the working and counter electrodes 188, 192, the target molecules 164 interact with the plurality of capture antibodies 196A-D positioned between the working and counter electrodes 188, 192. If the capture antibodies 196A-D corresponds to the target molecule 164, the target molecules 164 will bind to the capture antibodies 196A-D. If the target molecules 164 is bound to the capture antibody 196 and the target molecules 164 is also bound to one of the microparticles 128 through the detection antibody 160, the microparticle 128 is now positioned adjacent to the working and counter electrodes 188, 192. The second capillary valve 148 is eventually switched to the open state and the biological sample 120 flows away from the working and counter electrodes 188, 192, as shown in FIG. 3D. Then, the buffer fluid 124 clears any unbound microparticles 128 away from the working and counter electrodes 188, 192. Now, the clock signal generator 204 and the sinusoidal signal generator 208 are activated to apply a current to the working electrode 188. The applied current generates an electric field between the working electrode 188 and the microparticle counter electrode 192A and between the working electrode 188 and the reference counter electrodes 192B. The generated electric field is affected by the presence of microparticles 128. Specifically, the presence of the microparticle 128 adjacent to the microparticle counter electrode 192A will cause the measured current to be reduced. The microparticle current I_{cell} is converted to the microparticle voltage Vₘ by the microparticle transimpedance amplifiers 272A. The reference current Ir is converted to the reference voltage Vᵣ by the reference transimpedance amplifiers 272B. The IDC 212 receives the microparticle voltage Vₘ and the reference voltage Vᵣ and converts the impedance to voltage. The impedance is measured for each of the electrode subarrays 178A-D and the user can interpret the impedance signal for each electrode subarray 178A-D to determine a concentration of specific target molecule (biomarkers) in the biological sample 120.

### Experimental Data

An exemplary embodiment of the plasma separation membrane was manufactured and experimentally evaluated. Various aspects of the experiments are described below.

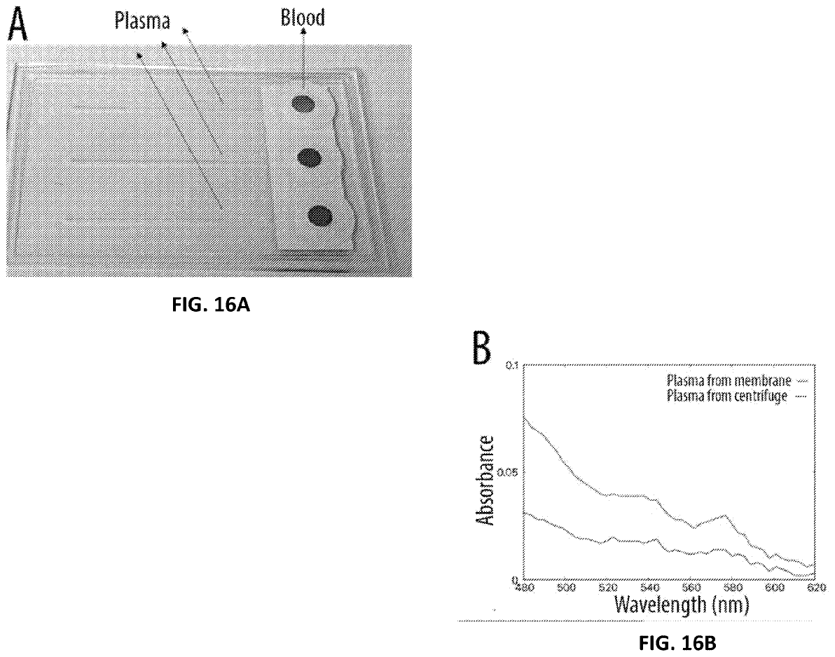

FIG. 16A shows a slide including three plasma separation membranes fluidly connected to three plasma wells. Each of the plasma separation membranes received 20 μL of blood and traveled through the membrane passively without introduction of an external force. As the blood travels through the plasma separation membrane, the plasma is separated from the blood and is entered into a corresponding plasma well. Additionally, plasma was separated from another 20 μL of blood using a centrifuge as control.

The separated plasma from the plasma separation membranes and the centrifuge were subjected to a UV-V spectroscopy analysis. The results of the UV-V spectroscopy analysis are shown in FIG. 16B. The troughs at 541 nm and 576 nm for the separated plasma from the plasma separated membranes shows the removal of red blood cells without the use of hemolysis. The plasma separated by the centrifuge also include troughs at 541 nm and 576 nm, which again shows the removal of red blood cells without the use of hemolysis.

For reasons of completeness, the following Clauses are provided.

In some aspects, the techniques described herein relate to a point-of-care diagnostic device including: an electronic component package; a complementary metal-oxide-semiconductor (CMOS) biosensor formed on the electronic component package, the CMOS biosensor including a fluidic system formed on the electronic component package, the fluidic system configured to transport fluid, the fluidic system including a first inlet configured to receive a buffer fluid, a second inlet configured to receive a biological sample, a reaction channel fluidly connected to the first inlet and the second inlet, wherein the buffer fluid and the biological sample flow through the reaction channel, and a waste bin fluidly connected to the reaction channel, the waste bin configured to receive the buffer fluid and the biological sample; an electrode array in fluid communication with the fluidic system, the electrode array configured to perform a multiplexing immunoassay procedure, the electrode array including a plurality of electrode pairs, the electrode array having a surface coating with an antibody; and an impedance detection circuit in electrical communication with the electrode array, wherein the impedance detection circuit is configured to detect an electrical impedance change caused by a binding of the biological sample to the antibody on each of the electrode pairs.

In some aspects, the techniques described herein relate to a point-of-care diagnostic device, wherein the electrode pairs include a working electrode and a counter electrode that are spaced apart by a gap.

In some aspects, the techniques described herein relate to a point-of-care diagnostic device, further including an inert electrode layer between the electrode array and the impedance detection circuit, wherein the inert electrode layer is composed of an inert metallic material.

In some aspects, the techniques described herein relate to a point-of-care diagnostic device, wherein the inert buffer layer is applied to the electrode array by an electroless nickel-gold plating method.

In some aspects, the techniques described herein relate to a point-of-care diagnostic device, wherein one of the plurality of electrode pairs includes a working electrode and a counter electrode, and wherein the working electrode is cross-shaped and the counter electrode is “T” shaped.

In some aspects, the techniques described herein relate to a point-of-care diagnostic device, wherein one of the plurality of electrode pairs includes a working electrode and a counter electrode, wherein the working electrode is hexagonally shaped and includes a hexagonal opening, and wherein the counter electrode is hexagonally shaped and positioned within the hexagonal opening.

In some aspects, the techniques described herein relate to a point-of-care diagnostic device, wherein one of the plurality of electrode pairs includes a working electrode and a counter electrode, wherein the electrode pair includes a working electrode and a counter electrode, wherein the working electrode is octagonally shaped and includes an octagonal opening, and wherein the counter electrode is octagonal shaped and positioned within the octagonal opening.

In some aspects, the techniques described herein relate to a point-of-care diagnostic device, the impedance detection circuit further includes a sinusoidal signal synthesizer.

In some aspects, the techniques described herein relate to a point-of-care diagnostic device, wherein the sinusoidal signal synthesizer is a direct digital frequency synthesizer.

In some aspects, the techniques described herein relate to a point-of-care diagnostic device, wherein the sinusoidal signal synthesizer is a frequency synthesizer with high order harmonics remover.

In some aspects, the techniques described herein relate to a point-of-care diagnostic device, wherein the impedance detection circuit is an impedance readout circuit.

In some aspects, the techniques described herein relate to a point-of-care diagnostic device, wherein the impedance readout circuit is an I/Q demodulator.

In some aspects, the techniques described herein relate to a point-of-care diagnostic device, wherein the impedance readout circuit is a polar demodulator.

In some aspects, the techniques described herein relate to a point-of-care diagnostic device, wherein the second inlet includes a porous membrane configured to store a plurality of microparticles and separate plasma from the biological sample.

In some aspects, the techniques described herein relate to a point-of-care diagnostic device, wherein the microparticles are coated in a secondary detection antibody configured to react with the biological sample.

In some aspects, the techniques described herein relate to a point-of-care diagnostic device, wherein the fluidic system further includes a buffer detour channel configured to receive the buffer fluid from the buffer channel, a first capillary valve ensuring that the buffer fluid and the biological sample enter the reaction channel at the same time, and a second capillary valve fluidly connected to the buffer detour channel and the reaction channel, the second capillary valve configured to allow exit of the buffer fluid and the biological sample from the reaction channel when both the buffer fluid from the buffer detour channel and the biological sample contact the second capillary valve.

In some aspects, the techniques described herein relate to a point-of-care diagnostic device, wherein the complementary metal-oxide-semiconductor (CMOS) biosensor is a dual in-line package composed of ceramic, wherein the dual in-line package includes at least 48 pins.

In some aspects, the techniques described herein relate to a point-of-care diagnostic device, wherein the electronic component package is no larger than 2 inches in any dimension.

In some aspects, the techniques described herein relate to a point-of-care diagnostic device including: an electronic component package; a complementary metal-oxide-semiconductor (CMOS) biosensor formed on the electronic component package, the CMOS biosensor including a fluidic system formed on the electronic component package, the fluidic system configured to transport fluid, the fluidic system including a first inlet configured to receive a buffer fluid, a second inlet configured to receive a biological sample, a reaction channel fluidly connected to the first inlet and the second inlet, wherein the buffer fluid and the biological sample flow through the reaction channel, and a waste bin fluidly connected to the reaction channel, the waste bin configured to receive the buffer fluid and the biological sample; an electrode array in fluid communication with the reaction channel, the electrode array configured to perform a multiplexing immunoassay procedure, the electrode array including a first electrode array section having a surface coating with a first capture antibody, a second electrode array section having a surface coating with a second capture antibody, a third electrode array section having a surface coating with a third capture antibody, and a fourth electrode array section having a surface coating with a fourth capture antibody; and an impedance detection circuit in electrical communication with the microelectrode array, wherein the impedance detection circuit is configured to detect an electrical impedance change caused by a binding of the biological sample to the antibody on each of the electrode pairs.

In some aspects, the techniques described herein relate to a point-of-care diagnostic device, wherein the first, second, third, and fourth electrode array sections include a 128 by 128 array of pairs of electrodes.

In some aspects, the techniques described herein relate to a point-of-care diagnostic device including: an electronic component package; a complementary metal-oxide-semiconductor (CMOS) biosensor formed on the electronic component package, the CMOS biosensor including a fluidic system formed on the electronic component package, the fluidic system configured to transport fluid, the fluidic system including a first inlet configured to receive a buffer fluid, a second inlet configured to receive a biological sample, a reaction channel fluidly connected to the first intake and the second inlet, wherein the buffer fluid and the biological sample mix in the reaction channel, and a waste bin fluidly connected to the reaction channel, the waste bin configured to receive the buffer fluid and the biological sample; an electrode array in fluid communication with the fluidic system, the electrode array configured to perform a multiplexing immunoassay procedure, the electrode array including a plurality electrode pairs, the electrode array having a surface coating with an antibody; an impedance detection circuit in electrical communication with the electrode array, wherein the impedance detection circuit is configured to detect an electrical impedance change caused by a binding of the biological sample to the antibody on each of the electrode pairs; and an impedance-to-digital-converter in electrical communication with the impedance detection circuit, wherein the impedance-to-digital converter is configured to convert a magnitude and a phase of impedance to a digital signal.

What is claimed is:

1. A point-of-care diagnostic device comprising:
an electronic component package;
a complementary metal-oxide-semiconductor (CMOS) biosensor formed on the electronic component package, the CMOS biosensor including
a fluidic system formed on the electronic component package, the fluidic system configured to transport fluid, the fluidic system comprising
a first inlet configured to receive a buffer fluid,
a second inlet configured to receive a biological sample,
a reaction channel fluidly connected to the first inlet and the second inlet, wherein the buffer fluid and the biological sample flow through the reaction channel, and
a waste bin fluidly connected to the reaction channel, the waste bin configured to receive the buffer fluid and the biological sample;
an electrode array in fluid communication with the fluidic system, the electrode array configured to perform a multiplexing immunoassay procedure, the electrode array including a plurality of electrode pairs, the electrode array having a surface coating with an antibody; and
an impedance detection circuit in electrical communication with the electrode array, wherein the impedance detection circuit is configured to detect an electrical impedance change caused by a binding of the biological sample to the antibody on each of the electrode pairs.

2. The point-of-care diagnostic device of claim 1, wherein the electrode pairs comprise a working electrode and a counter electrode that are spaced apart by a gap.

3. The point-of-care diagnostic device of claim 1, further comprising an inert electrode layer between the electrode array and the impedance detection circuit, wherein the inert electrode layer is composed of an inert metallic material.

4. The point-of-care diagnostic device of claim 3, wherein the inert buffer layer is applied to the electrode array by an electroless nickel-gold plating method.

5. The point-of-care diagnostic device of claim 1, wherein one of the plurality of electrode pairs comprises a working electrode and a counter electrode, and wherein the working electrode is cross-shaped and the counter electrode is “T” shaped.

6. The point-of-care diagnostic device of claim 1, wherein one of the plurality of electrode pairs comprises a working electrode and a counter electrode, wherein the working electrode is hexagonally shaped and includes a hexagonal opening, and wherein the counter electrode is hexagonally shaped and positioned within the hexagonal opening.

7. The point-of-care diagnostic device of claim 1, wherein one of the plurality of electrode pairs comprises a working electrode and a counter electrode, wherein the electrode pair comprises a working electrode and a counter electrode, wherein the working electrode is octagonally shaped and includes an octagonal opening, and wherein the counter electrode is octagonal shaped and positioned within the octagonal opening.

8. The point-of-care diagnostic device of claim 1, the impedance detection circuit further comprises a sinusoidal signal synthesizer.

9. The point-of-care diagnostic device of claim 8, wherein the sinusoidal signal synthesizer is a direct digital frequency synthesizer.

10. The point-of-care diagnostic device of claim 8, wherein the sinusoidal signal synthesizer is a frequency synthesizer with high order harmonics remover.

11. The point-of-care diagnostic device of claim 1, wherein the impedance detection circuit is an impedance readout circuit.

12. The point-of-care diagnostic device of claim 11, wherein the impedance readout circuit is an I/Q demodulator.

13. The point-of-care diagnostic device of claim 11, wherein the impedance readout circuit is a polar demodulator.

14. The point-of-care diagnostic device of claim 1, wherein the second inlet includes a porous membrane configured to store a plurality of microparticles and separate plasma from the biological sample.

15. The point-of-care diagnostic device of claim 14, wherein the microparticles are coated in a secondary detection antibody configured to react with the biological sample.

16. The point-of-care diagnostic device of claim 1, wherein the fluidic system further comprises
a buffer detour channel configured to receive the buffer fluid from the buffer channel,
a first capillary valve ensuring that the buffer fluid and the biological sample enter the reaction channel at the same time, and
a second capillary valve fluidly connected to the buffer detour channel and the reaction channel, the second capillary valve configured to allow exit of the buffer fluid and the biological sample from the reaction channel when both the buffer fluid from the buffer detour channel and the biological sample contact the second capillary valve.

17. The point-of-care diagnostic device of claim 1, wherein the complementary metal-oxide-semiconductor (CMOS) biosensor is a dual in-line package composed of ceramic, wherein the dual in-line package includes at least 48 pins.

18. The point-of-care diagnostic device of claim 1, wherein the electronic component package is no larger than 2 inches in any dimension.

19. A point-of-care diagnostic device comprising:
an electronic component package;
a complementary metal-oxide-semiconductor (CMOS) biosensor formed on the electronic component package, the CMOS biosensor including
a fluidic system formed on the electronic component package, the fluidic system configured to transport fluid, the fluidic system comprising
a first inlet configured to receive a buffer fluid,
a second inlet configured to receive a biological sample,
a reaction channel fluidly connected to the first inlet and the second inlet, wherein the buffer fluid and the biological sample flow through the reaction channel, and
a waste bin fluidly connected to the reaction channel, the waste bin configured to receive the buffer fluid and the biological sample;
an electrode array in fluid communication with the reaction channel, the electrode array configured to perform a multiplexing immunoassay procedure, the electrode array comprising
a first electrode array section having a surface coating with a first capture antibody,
a second electrode array section having a surface coating with a second capture antibody,
a third electrode array section having a surface coating with a third capture antibody, and
a fourth electrode array section having a surface coating with a fourth capture antibody; and
an impedance detection circuit in electrical communication with the microelectrode array, wherein the impedance detection circuit is configured to detect an electrical impedance change caused by a binding of the biological sample to the antibody on each of the electrode pairs.

20. The point-of-care diagnostic device of claim 19, wherein the first, second, third, and fourth electrode array sections comprise a 128 by 128 array of pairs of electrodes.

21. A point-of-care diagnostic device comprising:
an electronic component package;
a complementary metal-oxide-semiconductor (CMOS) biosensor formed on the electronic component package, the CMOS biosensor including
a fluidic system formed on the electronic component package, the fluidic system configured to transport fluid, the fluidic system comprising
a first inlet configured to receive a buffer fluid,
a second inlet configured to receive a biological sample,
a reaction channel fluidly connected to the first intake and the second inlet, wherein the buffer fluid and the biological sample mix in the reaction channel, and
a waste bin fluidly connected to the reaction channel, the waste bin configured to receive the buffer fluid and the biological sample;
an electrode array in fluid communication with the fluidic system, the electrode array configured to perform a multiplexing immunoassay procedure, the electrode array including a plurality electrode pairs, the electrode array having a surface coating with an antibody;
an impedance detection circuit in electrical communication with the electrode array, wherein the impedance detection circuit is configured to detect an electrical impedance change caused by a binding of the biological sample to the antibody on each of the electrode pairs; and
an impedance-to-digital-converter in electrical communication with the impedance detection circuit, wherein the impedance-to-digital converter is configured to convert a magnitude and a phase of impedance to a digital signal.
